(19) United States
(12) Patent Application Publication
(10) Pub. No.: US 2024/0358713 A1
(43) Pub. Date: Oct. 31, 2024

(54) PHARMCAEUTICAL COMPOSITION CONTAINING BENZOXAZEPINE DERIVATIVES, AND A METHOD FOR TREATING CANCER

(71) Applicant: Yarmouk University, Irbid (JO)

(72) Inventors: Firas Fandi Mahmud Awwadi, Irbid (JO); Shehadeh Abdelrahman A. Mizyed, Irbid (JO); Huda Adnan Salim Alsaadi, Irbid (JO); Khalid Mohammad Mahmoud Abu-Khadra, Irbid (JO); Areej Mashhour Tawfiq Assaf, Irbid (JO)

(73) Assignee: Yarmouk University, Irbid (JO)

(21) Appl. No.: 18/648,046

(22) Filed: Apr. 26, 2024

Related U.S. Application Data
(60) Provisional application No. 63/461,911, filed on Apr. 26, 2023.

(51) Int. Cl. A61K 31/553; A61K 9/16

(52) U.S. Cl. CPC A61K 31/553; A61K 9/1617; A61K 9/1652

(57) ABSTRACT

A pharmaceutical composition may include a benzoxazepine derivative compound or a pharmaceutically acceptable salt thereof, and a pharmaceutically acceptable carrier/excipient. A method for treating cancer may include administering a therapeutically effective amount of the compound of the pharmaceutical composition to a subject in need thereof.

### CROSS-REFERENCE TO EARLIER FILED APPLICATION

The present application claims priority from the U.S. provisional patent application No. 63/461,911 dated Apr. 26, 2023, which is incorporated herein by reference.

### TECHNICAL FIELD

The present disclosure relates to pharmaceutical compositions and methods for treating diseases related to tumor growth, and more particularly to, a pharmaceutical composition and a method of treatment of cancer by administering a therapeutically effective amount of the pharmaceutical composition to a subject.

### BACKGROUND INFORMATION

Cancer was the second-leading cause of worldwide death in 2020. Over the past years, there is an imperative requirement to synthesize efficient and safe chemotherapeutic agents that help inhibiting cancer cell proliferation.

It was recently reported in the art that the inhibition of pleotropic cytokine's interleukin IL-6 and tumor necrosis factor a (TNF-α) have important role in treating cancer. Development of such chemotherapeutic agents derived from benzoxazepine that have previously reported biological activities is significant. Many attempts in the art synthesize benzoxazepine derivatives.

Also, Mizyed et al. reported a synthesis procedure for benzoxazepine derivatives including the structure of the compound of the present disclosure in simple one-step reaction. The method is simply a condensation of 2-aminoethanol, 3-amino-1-propanol or 1,3-diamino propane with 2-(2-bromoethoxy)benzaldehydes in acetonitrile in the presence of anhydrous potassium carbonate as a base at reflux temperature.

The Spanish patent number ES2764497 discloses an inhibitor for phosphoinositide 3-kinase (PI3K)/Akt using benzoxazepine oxazolidinone derivatives that have activity against hyperproliferative disorders such as cancer.

The Russian patent number RU2654068 discloses benzoxazepine derivative compounds exhibiting anticancer activity, and more particularly compounds that inhibit PI3 kinase activity. There was also provided methods of using the compounds for the diagnosis or treatment of in vitro, in situ and in vivo mammalian cells, or for the treatment of concomitant pathological conditions, and their pharmaceutical compositions that can be used to treat diseases, conditions and/or disorders mediated by PI3 kinases.

### SUMMARY

It is an object of the present disclosure to provide a pharmaceutical composition comprising a benzoxazepine derivative and a method of treating cancer by administering a therapeutically effective amount of the pharmaceutical composition through inhibiting the release of Interleukin-6 (IL-6) and tumor necrosis α (TNF-α) in the cancer cells.

In aspects of the present disclosure, the pharmaceutical composition includes a compound of the general Formula (I), or a salt thereof, and a pharmaceutically acceptable carrier and/or excipient.

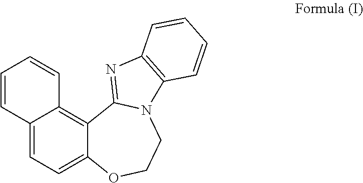

In some aspects of the present disclosure, the pharmaceutical composition may be used for treatment of cervical cancer, epithelial cancer, breast cancer, and/or colon cancer.

### BRIEF DESCRIPTION OF THE DRAWINGS

The disclosure will now be described with reference to the accompanying drawings, which illustrate embodiments of the present disclosure, without however restricting the scope of the disclosure thereto, and in which:

FIG. 1A illustrates the effect of the compound of the general Formula (I) configured in accordance with one or more embodiments of the present disclosure (50, 25, 10, 5 and 0.10 pg/mL concentrations) and cisplatin on the release of IL-6 of human cervical adenocarcinoma (“HeLa”) cell lines.

FIG. 1B illustrates the effect of the compound of the general Formula (I) configured in accordance with one or more embodiments of the present disclosure (50, 25, 10, 5 and 0.10 pg/mL concentrations) and cisplatin on the release of IL-6 cytokine of adenocarcinoma human alveolar basal epithelial cell lines (“A549”).

FIG. 1C illustrates the effect of the synthesized compound of the general Formula (I) configured in accordance with one or more embodiments of the present disclosure (50, 25, 10, 5 and 0.10 pg/mL concentrations) and cisplatin on the release of IL-6 cytokine of human breast cancer lines (“MCF-7”).

FIG. 1D illustrates the effect of the synthesized compound of the general formula (I) configured in accordance with one or more embodiments of the present disclosure (50, 25, 10, 5 and 0.10 pg/mL concentrations) and cisplatin on the release of IL-6 of Caucasian colon adenocarcinoma (“Caco-2”) cell lines.

FIG. 1E illustrates the effect of the synthesized compound of the general Formula (I) configured in accordance with one or more embodiments of the present disclosure (50, 25, 10, 5 and 0.10 pg/mL concentrations) and cisplatin on the release of IL-6 cytokine of normal fibroblast cell.

FIG. 2A illustrates the effect of the synthesized compound of the general Formula (I) configured in accordance with one or more embodiments of the present disclosure (50, 25, 10, 5 and 0.10 μg/mL concentrations) and cisplatin on the release of tumor necrosis TNF-α cytokine of HeLa cell lines.

FIG. 2B illustrates the effect of the synthesized compound of the general Formula (I) configured in accordance with one or more embodiments of the present disclosure (50, 25, 10, 5 and 0.10 μg/mL concentrations) and cisplatin on the release of TNF-α cytokine of A549 cell lines.

FIG. 2C illustrates the effect of the synthesized compound of the general Formula (I) configured in accordance with one or more embodiments of the present disclosure (50, 25, 10, 5 and 0.10 μg/mL concentrations) and cisplatin on the release of TNF-α cytokine of MCF-7 cell lines.

FIG. 2D illustrates the effect of the synthesized compound of the general Formula (I) configured in accordance with one or more embodiments of the present disclosure (50, 25, 10, 5 and 0.10 μg/mL concentrations) and cisplatin on the release of TNF-α cytokine of Caco-2 cell lines.

FIG. 2E illustrates the effect of the synthesized compound of the general Formula (I) configured in accordance with one or more embodiments of the present disclosure (50, 25, 10, 5 and 0.10 μg/mL concentrations) and cisplatin on the release of TNF-α cytokine of normal fibroblast cell.

FIG. 3A illustrates a chart showing the H¹-NMR spectrum for the of the general Formula (I) in (CDCl₃, 1% TMS), wherein such spectrum is obtained using Bruker Avance (III) 400 MHz instrument.

FIG. 3B illustrates a chart showing the C¹³-NMR spectrum for the of the general Formula (I) in (CDCl₃, 1% TMS), wherein such spectrum is obtained using Bruker Avance (III) 400 MHz instrument.

FIG. 3C illustrates a chart showing the ¹³C-dept-135 NMR spectrum for the of the general Formula (I) in (CDCl₃, 1% TMS), wherein such spectrum is obtained using Bruker Avance (III) 400 MHz instrument.

FIG. 4 illustrates a high-resolution mass spectrum (“HR-MS”) of the general Formula (I) on ([M+H+]⁺) using electrospray ion trap (“ESI”) technique by collision-induced dissociation on a Bruker APEX-IV (7 Tesla) instrument.

FIG. 5 illustrates a crystal structure collected on a Xcalibur, Eos diffractometer of of the general Formula (I) configured in accordance with one or more embodiments of the present disclosure.

### DETAILED DESCRIPTION

Embodiments of the present disclosure provide a pharmaceutical composition including the compound of the of the general Formula (I) or a pharmaceutically acceptable salt thereof and a pharmaceutically acceptable carrier/excipient, and a method for treating cancer including administering a method of treatment of cancer including administering a therapeutically effective amount of the pharmaceutical composition to a subject.

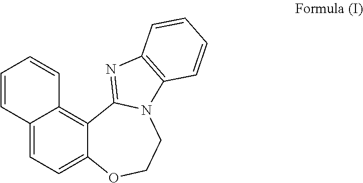 [SMILES: c1ccc2c3c(ccc2c1)OCCn1c-3nc2ccccc21]

In embodiments of the present disclosure, the cancer may include cervical cancer, epithelial cancer, breast cancer, and/or colon cancer.

In embodiments of the present disclosure, the cancer may be caused by HeLa cell lines, A549 cell lines, MCF-7 cell lines, and/or Caco-2 cell lines.

The pharmaceutical composition of the present disclosure affects the release of IL-6 and TNF in which the compound of the general formula (I) stimulated the release of IL-6 in all cancer cells tested. On the other hand, only 50 μg/mL concentration was able to stimulate Caco-2 cell lines to significantly release TNF-α.

All components of the pharmaceutical composition have to be pharmaceutically acceptable. The term “pharmaceutically acceptable” means at least non-toxic.

The term “pharmaceutical composition”, as used herein, is intended to include a pharmaceutical active compound of general formula (I) and/or a pharmaceutically acceptable salt thereof.

The pharmaceutical composition can be, for example, in a liquid form, e.g., a solution, syrup, emulsion and suspension, or in a solid form, e.g., a capsule, caplet, tablet, pill, powder and suppository. Granules, semi-solid forms and gel caps are also considered. In case that the pharmaceutical composition is a liquid or a powder, dosage unit optionally is to be measured, e.g., in the dosage unit of a teaspoon.

The pharmaceutical composition of this disclosure can be formulated for oral administration in solid or liquid form, for parenteral injection or for rectal administration. The pharmaceutical composition can be administered to humans and other mammals orally, sublingually, rectally, pareneterally, intracisternally, intraurethrally, intraperitoneally, topically (as powder, ointment or drop), buccally or as an oral or nasal spray. The term “parenterally”, as used herein, refers to modes of administration which include intravenous, intramuscular, intraperitoneal, subcutaneous, intra-articular injection and infusion.

Pharmaceutical compositions of this disclosure for parenteral injection comprise pharmaceutically acceptable sterile aqueous or non-aqueous solutions, dispersions, suspensions or emulsions and sterile powders for reconstitution into sterile injectable solutions or dispersions. Examples of suitable aqueous and non-aqueous carriers, diluents, solvents or vehicles include water, ethanol, polyols (propylene glycol, polyethylene glycol, glycerol, and the like), suitable mixtures thereof, vegetable oils (such as olive oil) and injectable organic esters such as ethyl oleate. Proper fluidity may be maintained, for example, by the use of a coating such as lecithin, by the maintenance of the required particle size in the case of dispersions, and by the use of surfactants.

The term “pharmaceutical acceptable carrier”, as used herein, means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; starches such as corn starch; cellulose and its derivatives such as sodium carboxymethyl cellulose, ethyl cellulose and cellulose acetate; powdered tragacanth; binding agents such as hypromellose; disintegrating agents such as crosscarmellose; water; malt; gelatin; talc; excipients such as cocoa butter and suppository waxes; oils such as peanut oil; cottonseed oil; safflower oil, sesame oil, olive oil, corn oil and soybean oil; glycols, such as propylene glycol; esters such as ethyl oleate and ethyl laurate; agar; buffering agents such as magnesium hydroxide and aluminium hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgement of the formulator.

All components of the pharmaceutical composition have to be pharmaceutically acceptable. The term “pharmaceutically acceptable” means at least non-toxic. The therapeutically active component should preferably be present in the above-mentioned pharmaceutical composition, the concentration of about 0.1 to 99.5% by weight, preferably of about 0.5 to 95% by weight of the total mixture.

The above-mentioned pharmaceutical composition can further contain other pharmaceutical active compounds in addition to the compound of general formula (I) according to the disclosure.

The disclosure will be further illustrated on the basis of examples and a detailed description from which further features and advantages may be taken. It is to be noted that the following explanations are presented for the purpose of illustrating and description only; they are not intended to be exhaustive or to limit the disclosure to the precise form disclosed.

### Example 1

### Synthesis of the Compound of the General Formula (I)

The tested compound of the General Formula (I) is prepared according to (mizyad et al) method of preparation as shown in the following preparation scheme:

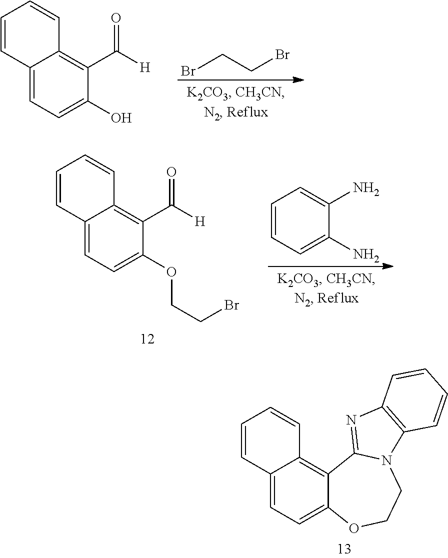

In a 250 mL three-necked flask with a magnetic stirrer bar and a reflux condenser under a nitrogen atmosphere, a 0.49 g (3.6 mmol) of anhydrous K₂CO₃ was suspended in 100 mL of anhydrous acetonitrile CH₃CN. To this solution, with stirring at reflux temperature, a solution of aldehyde (1.8 mmol) in 50 mL dry CH₃CN and a solution of diamine (1.8 mmol) in 50 mL of dry CH₃CN were dropwise added simultaneously over a period of 12 h. The reaction mixture was further refluxed by stirring overnight. The reaction mixture was filtered, and the solvent was evaporated. The crude product was purified by chromatographic silica gel using eluent hexane: ethyl acetate (8.5:1.5) to give a brown solid yield (0.28 g, 54%). M.p is (142-144° C.).

### Example 2

### Characterization of the Compound of the General Formula (I)

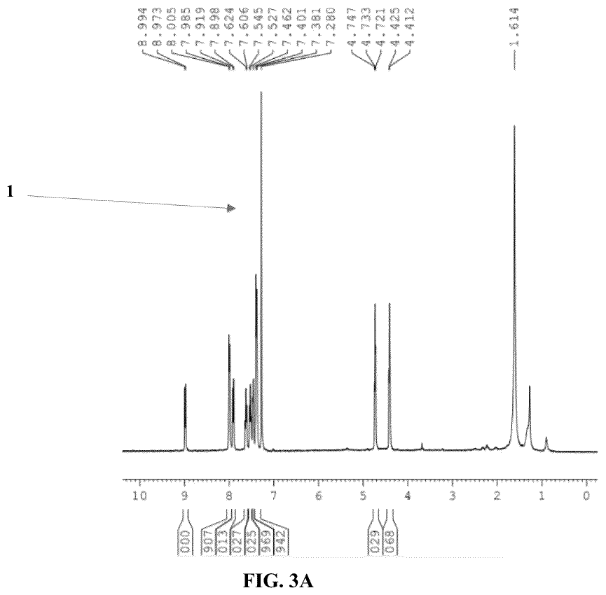

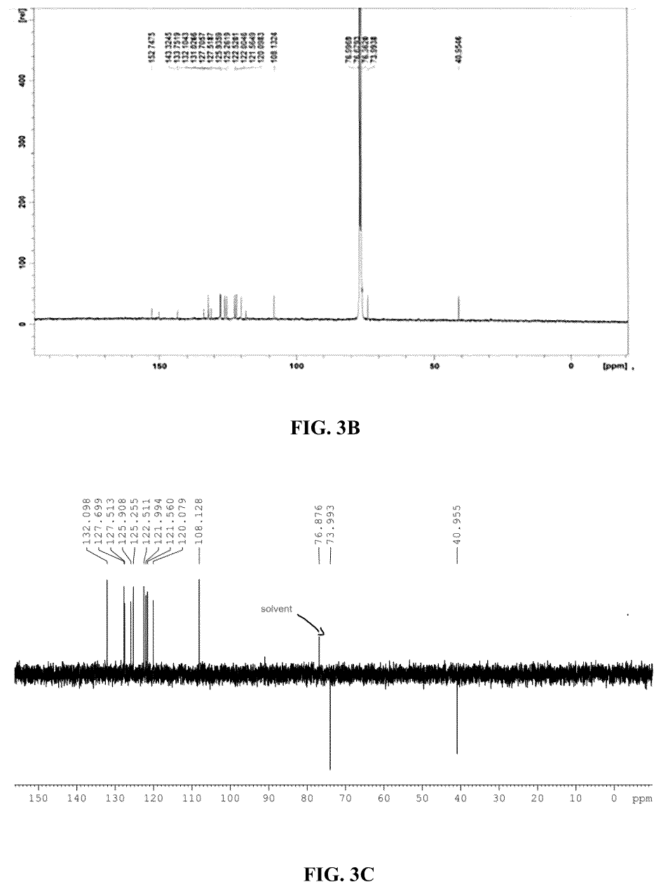

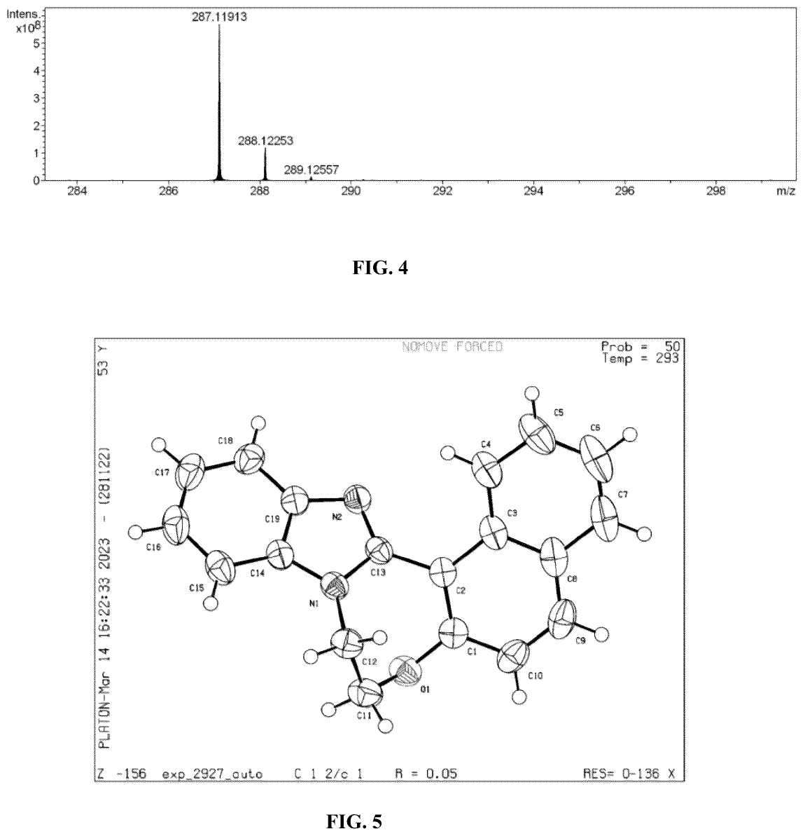

Reference is now being made to FIGS. 3A-3C, 4, 5.

The H¹, C¹³ and dept-135 spectrum for the tested compound of formula 1. Spectra were recorded on 400 MHz and 100 MHz for C¹³ and H¹ respectively, using Bruker Avance (III) 400 MHz. Chemical shifts were recorded using (CDCl₃, 1% TMS) or C₂D₆SO (d₆-DMSO) as solvents.

As illustrated in FIGS. 3A and 3B, ¹H NMR spectra of tested compound of the present disclosure have identical two-triplet patterns in the chemical shift range of δ≈4.4-4.6 ppm, assigned for the CH₂—CH₂ groups within the seven-membered ring. The evidence that a N—C≡N group is formed is proofed by the disappearance of CHNN segment signals in which are usually appear around 4.6 ppm in the ¹H-NMR and 79 ppm in the ¹³C-NMR spectra. Also, The NMR spectral data reveals the disappearance of the aldehyde carbonyl signal at about 200 ppm in the ¹³C-NMR spectrum and at about 9 ppm for the aldehyde proton in the ¹H-NMR spectrum. In addition, ¹H-NMR spectrum showed clearly the presence of the methylene groups at 4.44 and 4.54 ppm, and the ¹³C-NMR spectrum also showed the presence of methylene groups at 47.3 and 68.8 ppm.

As illustrated in FIGS. 3C, DEPT results give the appropriate CH, CH₂, and CH₃ signals for the compound of formula 1.

A selected bond distances and angles from the X-ray crystal are listed in Table 2. The X-ray crystal structure reveal the C═N double bond character as the bond distance is 1.32 for N₂-C₁₃ is shorter than other N₂—C bond distances. As shown in FIG. which confirm the proposed structure of the inhibitor of the present disclosure.

TABLE 1
selected bond distance and angles of
the compound of the General Formula (I)
Compound of the General Formula (I)

| | |
|---|---|
| N1—C13 | 1.3730(18) |
| N2—C13 | 1.3194(19) |
| N1—C14 | 1.377(2) |
| N2—C19 | 1.389(2) |
| O1—C1 | 1.3858(19) |
| O1—C11 | 1.429(2) |
| C2—C13 | 1.468(2) |
| C13—N1—C12 | 123.66(13) |
| N1—C12—C11 | 111.24(13) |
| C12—C11—O1 | 112.97(14) |
| O1—C1—C2 | 120.34(14) |
| C1—O1—C11—C12 | 44.2(2) |
| O1—C11—C12—N1 | 40.7(2) |
| C11—C12—N1—C13 | 69.28(19) |
| C12—N1—C13—C2 | 3.2(2) |
| N1—C13—C2—C1 | 43.84(19) |
| C13—C2—C1—O1 | 4.6(2) |
| C2—C1—O1—C11 | 75.53(18) |

### Example 3

### Evaluation of the Anti-Proliferative Activity

### Cell Lines and Cultivation Conditions

The benxoxazepine derivative compound of the present disclosure was dissolved in DMSO to give a 20 μg/L concentration. The DMSO concentration was less than 1.0% in all experiments and controls.

The evaluation of anti-proliferative activity was preformed to different cancer cells named human cervical adenocarcinoma (HeLa), adenocarcinoma human alveolar basal epithelial cells (A549), human breast cancer (MCF-7), Caucasian colon adenocarcinoma Caco-2, compared with human fibroblast cell lines (F2). All cells were grown in Dulbecco's modified Eagle's minimal essential medium (DMEM), supplemented with 25 mM glucose, 10% inactivated fetal bovine serum (Flow, McLean, VA, USA), and 1% penicillin/streptomycin, and maintained at 37° C. in a humidified incubator with 5% CO₂. The cells with 30-40 passages were used for further investigations.

### Cytotoxic Activity (MTT Assay) and IC₅₀ Values

Exponentially growing cells A549, HeLa, Caco-2, MCF-7, and normal fibroblast cell cells have been washed and seeded at 3×10³ cells/well (100 μL/well) in 96 well microplates (Greiner, Germany). After 24 h, cells were confluent, and media was changed, and cells were incubated with the compound of the present disclosure (200-1 μg/mL) for 24 h. In vitro anti-proliferative activity was measured by the cell growth inhibition assay using 3-(4,5-dimethyl-thiazol-2-yl)-2,5-di-phenyltetrazolium bromide (MTT) assay according to the manufacturer's instructions (Promega Corporation, Madison, WI). Briefly, the assay measured the formation of blue formazan product as a result of the reduction of MTT by mitochondrial dehydrogenase, which indicates the normal function of mitochondria and cell viability. The amount of formazan was quantified using a microplate reader (Thermo Fisher Scientific, Waltham, MA, USA) and compared to the optical density obtained for the control (untreated) at 570 nm. The IC₅₀ values were calculated according to the equation for Boltzmann sigmoidal concentration response curve using the nonlinear regression models (GraphPad, Prism Version 7, San Diego, CA, USA). The average of three replicates of IC₅₀ values were calculated and mentioned in the present disclosure Table 1.

The anti-proliferative activity of the investigated compound of formula 1 of the present disclosure was expressed in IC₅₀ values (the concentration needed to inhibit 50% of cell growth) compared to untreated cells (negative control). Screening cytotoxic activity and IC₅₀ values was determined as shown in Table 2.

A comparison of the tested compound of the present disclosure with cisplatin was investigated. Results showed proliferation inhibition with IC₅₀ values between 0.003 μg/mL and 41.27 μg/mL for the compound, while the reference anti-proliferative drug cisplatin was (0.13-3.99 μg/mL). HeLa cells had the lowest IC₅₀ values with high inhibitory effect, while the highest for Caco-2 cell lines when cells were treated with compound of the present disclosure and cisplatin.

TABLE 2
The IC₅₀ of the compound for the different cell lines and the control drug.

| | Cells | | | | |
|---|---|---|---|---|---|
| Compound | A549 | HeLa | Caco-2 | MCF-7 | Fibroblast |
| Benzoxazepine | 15.65 ± 0.54 | 0.003 ± 0.001 | 41.27 ± 2.85 | 17.58 ± 0.72 | 19.87 ± 1.40 |
| derivative | | | | | |
| Compound | | | | | |
| Cisplatin | 3.13 ± 0.53 | 0.13 ± 0.02 | 3.99 ± 0.67 | 1.51 ± 0.10 | 2.83 ± 0.12 |
| DMSO % | 0.33 ± 0.05 | 0.15 ± 0.02 | 23.40 ± 3.20 | 0.097 ± 0.02 | 0.13 ± 0.02 |

Selectivity index (SI) was determined based on the following formula:

$$SI = \frac{{IC}_{50} for fibroblast}{{IC}_{50} for cancel cell}$$

The calculated SI values for the compound of the general Formula (I) for cancer cell tested A549, HeLa, Caco-2, and MCF-7 were 1.27, 7332.1, 0.48, and 1.13 respectively.

If the selectivity index (SI) values higher than 1.0 it may be considered anticancer specificity.

If the selectivity index (SI) values higher than 1.0 it may be considered selective to cancer cell.

Therefore, the compound of the general Formula (I) has very high selectivity to HeLa cell line.

The compound of the general Formula (I) may be considered for anticancer treatment for A549, HeLa, Caco-2, and/or MCF-7 tumors.

### Example 4

### Effect of The Compound of the General Formula (I) on the Production of IL-6 and TNF-α

The effect of the compound of the general Formula (I) was tested in vitro, 1×10⁶ cells/mL of each A549, HeLa, Caco-2, and MCF-7 cancer cell lines were seeded in a 24-well plate and either left for 60 minutes before the treatment with the inhibitor and the control drug, cisplatin. Cells were then treated with different concentration particularly at 1, 5, 10, 25, and 50 μg/mL separately in a growth medium or left without treatment (control). Cultures were incubated in a humidified atmosphere of 37° C. and 5% CO₂ overnight. Supernatants obtained from controls and treated cells were harvested for analysis by an enzyme-linked immunosorbent assay (ELISA). Non-treated cells were used as negative controls. The concentrations of IL-6 and TNF-α cytokines (in 100 μL of cancer cell lines' supernatants) were determined by ELISA Ready-SET-Go e-Bioscience kit according to the manufacturer's protocol (Bioscience, San Diego, USA). All incubation steps were performed at room temperature. The optical density at 450 nm, corrected by the reference wavelength 570 nm, was measured with a microplate reader (Biotek, Winooski, VT, USA). All cytokine assays were calibrated against the World Health Organization's international standards by the kit's manufacturer. The lower limit of detection for the individual assays for human IL-6 was 2 μg/ml and 4 μg/ml for TNF-α.

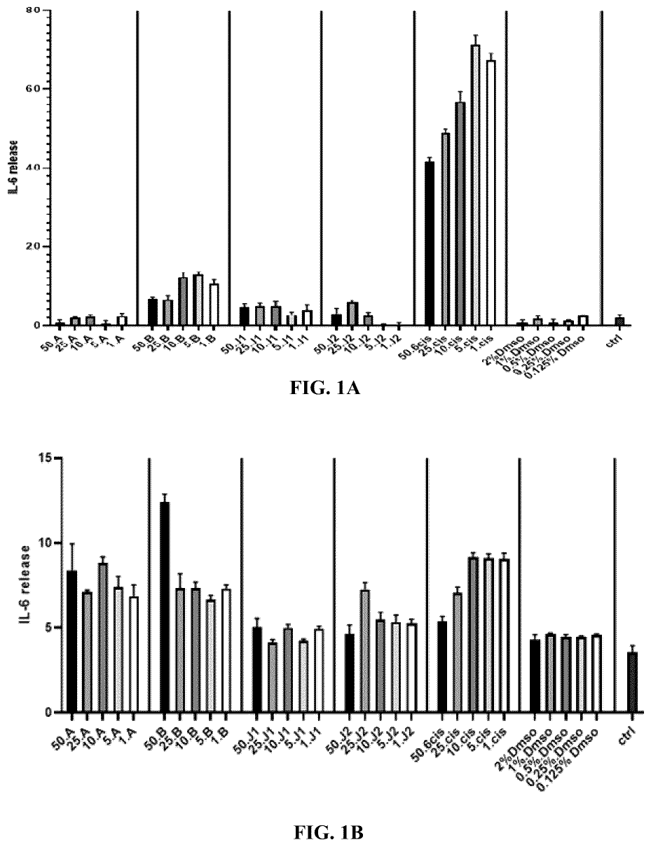

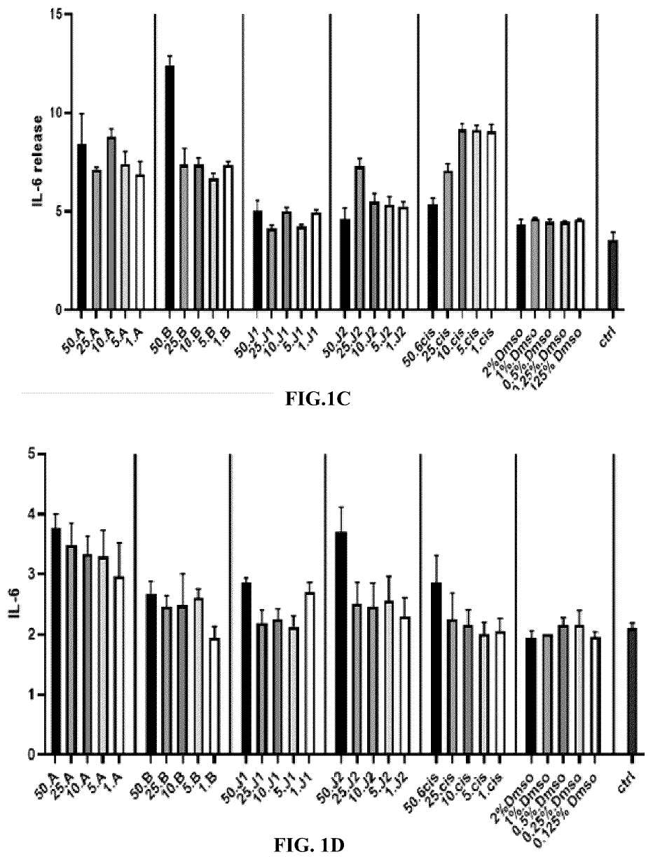

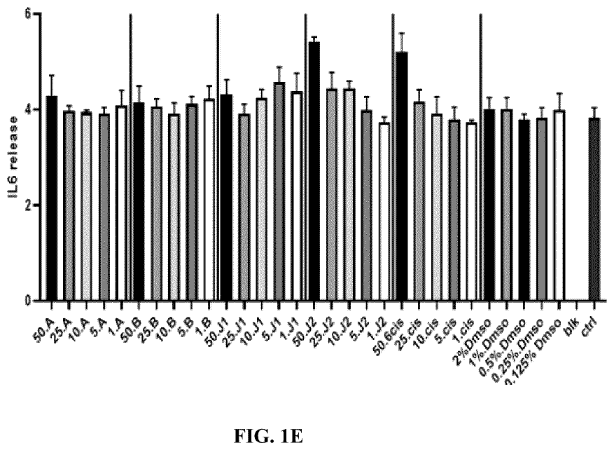

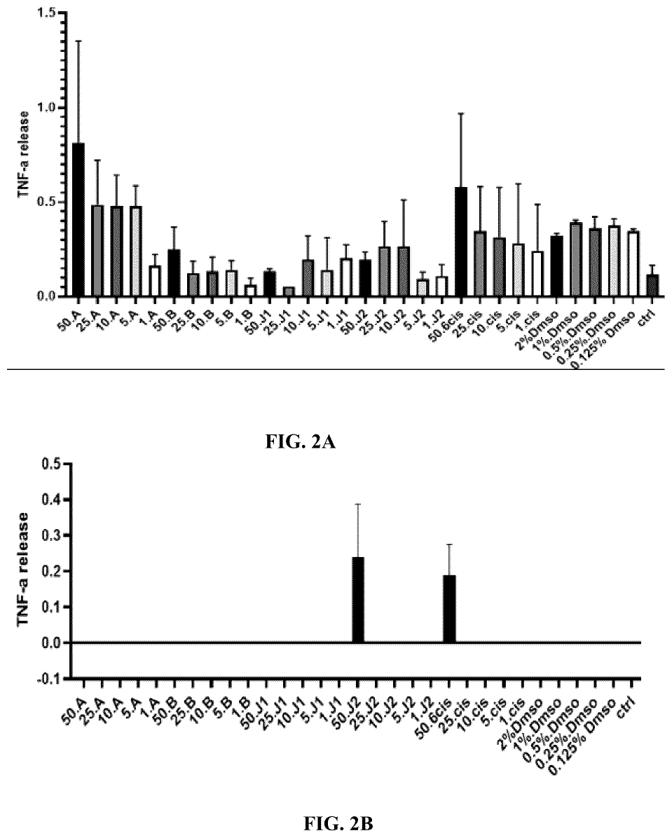

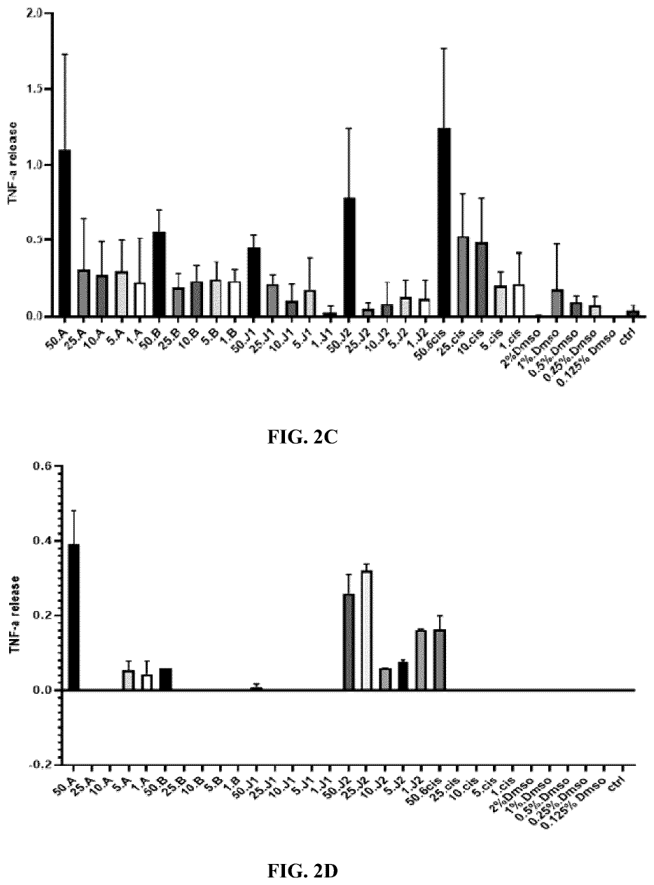

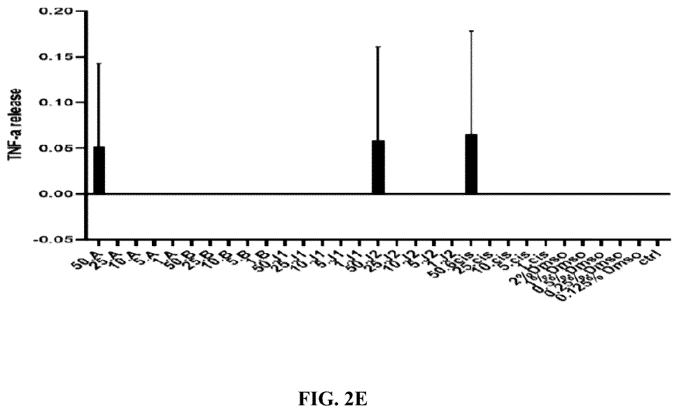

FIGS. 1A-1D, 2A-2D illustrate the treated four cancer cell lines A549, HeLa, Caco-2, and MCF-7 with different concentrations of the tested compound of the present disclosure particularly 50, 25,10, 5 and 1 μg/mL indicate the levels of human Interleukin-6 (IL-6) and tumor necrosis factor a (TNF-α) pro-inflammatory cytokines. The inflammatory process was found to significantly play a role in tumorigenesis with increasing evidence that pro-inflammatory cytokines like interleukin-6 and tumor necrosis factor-α are involved in the development of cancer.

The effect of the compound of compound of the general Formula (I) on the release of IL-6 and TNF-α was compared with cisplatin control drug as illustrated in FIGS. 1A-1E. the tested compound was able to stimulate the release of IL-6 in A549, HeLa, and MCF-7 when treated with all concentrations of inhibitor of the present disclosure. However, no effect was noticed on Caco-2 and normal fibroblast cells. On the other hand, the control drug, cisplatin was only able to induce the release of IL-6 for Caco-2 cell line.

FIGS. 2A-2E illustrate the effect of the tested compound of the present disclosure and cisplatin on TNF-α cytokine as it showed variable effects depending on cancer cell type as it either induced as pro- or anti-tumorigenic. The tested compound of was able to stimulate Caco-2 cell lines to significantly release TNF-α when treated with 50 μg/mL concentration only. While, the control drug release TNF-α for A459, HeLa and MCF-7 cancer cell lines.

In some embodiments, the tested compound of the present disclosure and the control drug was not able to induce the release of TNF-α and IL-6 from the normal fibroblast cells.

### Example 5

### Formulation of the Pharmaceutical Composition

The compound of the formula (I) and various amounts of hypromellose acetate succinate-MG polymer is used to generate an amorphous solid dispersion intermediate. The compound of the formula (I) amorphous solid dispersion and hypromellose acetate succinate (“HPMCAS”) (50%/50%, w/w) are weighed and dissolved in dimethyl sulfoxide (“DMSO”) and spray dried using a suitable amorphous solid dispersion spray drying processing parameters to produce an amorphous compound-hypromellose HPMCAS. The resulted compound-HPMCAS amorphous solid dispersion is mixed with microcrystalline cellulose, crosscarmellose sodium, about 1% w/w sodium lauryl sulfate, about 2% w/w colloidal silicon dioxide, and about 1.5% w/w of magnesium stearate in a suitable blender. After that, intragranule (The intra-granule blend is roller compacted and the compacted material is sized to produce granules) blending and dry granulation/Sizing were carried out. Then, the granules with suitable excipients are weighed and sieved for blending.

Success criteria to manufacturing a batch with reasonable yield (>60%), low residual solvents (53000 ppm), as well as meeting specifications for assay and purity testing.

While embodiments of the present disclosure have been described in detail and with reference to specific embodiments thereof, it will be apparent to one skilled in the art that various additions, omissions, and modifications can be made without departing from the spirit and scope thereof.

What is claimed is:

1. A pharmaceutical composition comprising a compound of the general formula (I) or a pharmaceutically acceptable salt thereof, and a pharmaceutically acceptable carrier/excipient.

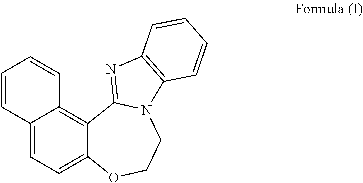

2. The pharmaceutical composition of claim 1, wherein the composition is in oral dosage form.

3. The pharmaceutical composition of claim 2, wherein the oral dosage form comprises liquid oral dosage form.

4. The pharmaceutical composition of claim 3, wherein the liquid oral dosage form comprises emulsion, solution, suspension, syrup or elixir.

5. The pharmaceutical composition of claim 2, wherein the oral dosage form comprises solid oral dosage form.

6. The pharmaceutical composition of claim 5, wherein the solid oral dosage form comprises tablet, coated tablet, powder, powder for reconstitution, pellets, beads, mini-tablet, multilayer tablet, bilayered tablet, tablet-in-tablet, pill, micro-pellet, small tablet unit, multiple unit pellet system, disintegrating tablet, dispersible tablet, granules, microspheres, multiparticulates, capsule, sachet, or sprinkles.

7. The pharmaceutical composition of claim 2, wherein the composition is for parenteral injection.

8. The pharmaceutical composition of claim 2, wherein the composition is for rectal administration.

9. The pharmaceutical composition of claim 2, wherein the composition is formulated in delayed release formulation.

10. The pharmaceutical composition of claim 2, wherein the composition is formulated in sustained release formulation.

11. The pharmaceutical composition of claim 2, wherein the composition is formulated in targeted release formulation.

12. The pharmaceutical composition of claim 1, wherein the composition inhibits the release of Interleukin-6 cytokine (IL-6) in cancer cells.

13. The pharmaceutical composition of claim 1, wherein the composition stimulates the release of tumor necrosis α factor (TNF-α) in cancer cells.

14. A method for treating cancer comprising administering a therapeutically effective amount of the pharmaceutical composition of claim 1 to a subject.

15. The method of claim 14, wherein the cancer is cervical cancer, epithelial cancer, breast cancer, and/or colon cancer.

16. The method of claim 15, wherein the cancer is caused by HeLa cell lines, A549 cell lines, MCF-7 cell lines, and/or Caco-2 cell lines.
